(19) United States
(12) Patent Application Publication
(10) Pub. No.: US 2013/0337063 A1
(43) Pub. Date: Dec. 19, 2013

(54) PHARMACEUTICAL COMPOSITIONS OF MARAVIROC AND PROCESS FOR THE PREPARATION THEREOF

(71) Applicants: Bandi Parthasaradhi Reddy, Hyderabad (IN); Podili Khadgapathi, Hyderabad (IN); Goli Kamalakar Reddy, Hyderabad (IN); Nelluri Rama Rao, Hyderabad (IN)

(72) Inventors: Bandi Parthasaradhi Reddy, Hyderabad (IN); Podili Khadgapathi, Hyderabad (IN); Goli Kamalakar Reddy, Hyderabad (IN); Nelluri Rama Rao, Hyderabad (IN)

(73) Assignee: HETERO RESEARCH FOUNDATION, HYDERABAD, ANDHRA PRADESH (IN)

(21) Appl. No.: 13/981,417

(22) PCT Filed: Feb. 17, 2012
(86) PCT No.: PCT/IN2012/000115

§ 371 Date: Jul. 24, 2013

(30) Foreign Application Priority Data
Feb. 11, 2011 (IN) 491/CHE/2011

(51) Int. Cl. A61K 31/46; A61K 9/20

(52) U.S. Cl. CPC A61K 31/46; A61K 9/2018; A61K 9/2054; A61K 9/2059; A61K 9/2095
USPC 424/474; 514/304; 424/489

(57) ABSTRACT

The present disclosure relates to solid dosage forms comprising the CCR5 co-receptor antagonist maraviroc. More particularly, the present disclosure relates to a solid oral dosage form containing maraviroc which has favorable disintegration properties.

### PRIORITY

This patent application claims priority to Indian application number 491/CHE/2011, filed on Feb. 21, 2011, the contents of which are incorporated by reference herein in their entirety.

### FIELD OF THE DISCLOSURE

The present disclosure relates to solid dosage forms comprising maraviroc, a CCR5 co-receptor antagonist. More particularly, the present disclosure relates to solid oral dosage forms containing maraviroc, and process for preparing the same.

### BACKGROUND OF THE DISCLOSURE

Maraviroc is chemically, N-{(1S)-3-[3-(3-isopropyl-5-methyl-4H-1,2,4-triazol-4-yl)-exo-8-azabicyclo[3.2.1]oct-8-yl]-1-phenylpropyl}-4,4 difluoro cyclohexane carboxamide, having the following structural formula:

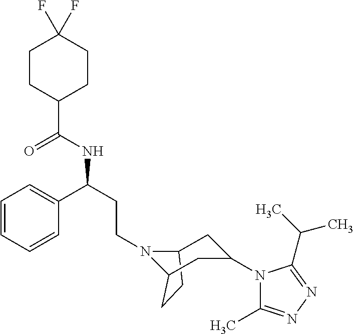 [SMILES: Cc1nnc(C(C)C)n1C1CC2CCC(C1)N2CC[C@H](NC(=O)C1CCC(F)(F)CC1)c1ccccc1]

Maraviroc is a modulator of the chemokine receptor CCR5 and is useful in the treatment of retroviral diseases caused by viruses that utilize CCR5 to enter cells. In particular, maraviroc has been disclosed as a useful therapeutic agent in the treatment of HIV, a retroviral infection genetically related to HIV AIDS, and also inflammatory disease.

Maraviroc is marketed under the trade name Selzentry® in United States by ViiV Healthcare in the form of oral tablet.

Maraviroc and its pharmaceutically acceptable salts and solvates thereof are disclosed in U.S. Pat. No. 6,667,314.

U.S. Pat. No. 7,576,097 assigned to Pfizer discloses crystalline Form A and Form B of maraviroc.

What is needed are improved pharmaceutical compositions containing maraviroc as an active agent.

### SUMMARY

Described herein are solid oral compositions comprising maraviroc and a pharmaceutically acceptable excipient, prepared either by direct compression or dry granulation process.

In one aspect, described herein is a solid oral composition comprising maraviroc, a diluent, a disintegrant and colloidal silicon dioxide as a dispersing agent, wherein the solid oral composition is prepared either by direct compression or dry granulation by slugging.

Another aspect provides disintegration time of the composition is less than 2 minutes when measured by a USP disintegration apparatus at 37° C.±2° C.

In another aspect, a solid oral composition in the form of a tablet comprises i) 20 wt % to 30 wt % of amorphous maraviroc having a particle size d₉₀ not more than 150 μm; ii) 0.5 wt % to 5 wt % of the colloidal silicon dioxide as a dispersing agent; iii) 40 wt % to 80 wt % of the diluent; and iv) 1 wt % to 8 wt % of the disintegrant based on the total weight of the composition.

In yet another aspect, a tablet composition comprises maraviroc, microcrystalline cellulose, lactose, colloidal silicon dioxide, magnesium stearate and one or more disintegrants selected from croscarmellose sodium, sodium starch glycolate, crospovidone and polacrilin potassium; where in the tablet is prepared by either direct compression or dry granulation by slugging.

In a further aspect, a process for the preparation of tablet composition comprising maraviroc and a pharmaceutically acceptable excipient comprises: (i) dry mixing maraviroc with a diluent, a disintegrant and colloidal silicon dioxide, (ii) blending the dry mix to form a blend, (iii) lubricating the blend and finally (iv) compressing the lubricated blend of step (iii) into tablets.

In another aspect, a process for preparing a compressed tablet composition comprising maraviroc comprises (a) sifting and blending the maraviroc with a diluent, a disintegrant and colloidal silicon dioxide to form a blended mixture, (b) compressing the blended mixture of step (a) to form slugs, (c) sizing the slugs to form granules; (d) blending the granules with an excipient selected from a binder, a lubricant and a glidant; and (e) compressing the granules of step (d) in to tablets.

In another aspect, included herein is a solid oral composition comprising amorphous maraviroc having a particle size d₉₀ not more than 150 μm, preferably 5-100 μm, more preferably 10-80 μm.

In yet another aspect, included herein is a method of improving patient compliance in a patient in need of treatment of HIV-1 comprising administering the solid oral dosage forms disclosed herein.

### DETAILED DESCRIPTION

Described herein are novel maraviroc solid oral dosage forms that have favorable disintegration and dissolution characteristics. The inventors of the present application have found that by using a combination of a disintegrant and a particular dispersing agent, tablets having favorable disintegration times and dissolution characteristics can be produced, thereby providing bioequivalent compositions. Further, inventors of the present invention have surprisingly found that amorphous maraviroc having a particle size d₉₀ less than 150 μm was found to exhibit excellent in-vitro and in-vivo characteristics that were also found to be comparable with the marketed formulation.

As used herein, term “maraviroc” includes maraviroc in the form of a free base or its pharmaceutically acceptable salts, amorphous maraviroc, crystalline maraviroc and any isomers, hydrate and solvates thereof.

Disclosed herein are solid oral compositions comprising maraviroc, a diluent, a disintegrant and a colloidal silicon dioxide as dispersing agent. The compositions optionally further comprise binders, lubricants and glidants.

Solid oral compositions include tablets, capsules, and granules.

A “pharmaceutical composition” comprises an active pharmaceutical ingredient and a pharmaceutically acceptable excipient. The term “pharmaceutically acceptable excipient” includes a pharmaceutically acceptable material, composition or vehicle, suitable for administering an active pharmaceutical ingredient. Each excipient should be “acceptable” in the sense of being compatible with the other ingredients of the formulation and not injurious to the patient. Excipients include diluents, binders, disintegrants, glidants, lubricants and others.

Exemplary diluents (also called fillers) include lactose, sugar, starches, modified starches, mannitol, sorbitol, inorganic salts, cellulose derivatives (e.g., microcrystalline cellulose), calcium sulfate, xylitol, lactitol, starch, pregelatinized starch, kaolin, sucrose, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide, and the like, and mixtures thereof.

The term “disintegrant” as used herein means a compound used in solid dosage forms to promote the disruption of the solid mass into smaller particles which are more readily dispersed or dissolved. Suitable disintegrants include, by way of example and without limitation, polacrillin potassium, croscarmellose sodium, crospovidone (e.g., KOLLIDON®, POLYPLASDONE®), polyvinylpyrrolidone, sodium starch glycolate (e.g., PRIMOGEL, EXPLOTAB®), hydroxypropylmethylcellulose, hydroxypropyl cellulose, carboxymethylcellulose calcium, starches such as corn starch, potato starch, pre-gelatinized starch and modified starches, clays, bentonite, microcrystalline cellulose (e.g., Avicel™), carsium (e.g., Amberlite™), alginates, gums such as agar, guar, locust bean, karaya, pectin, tragacanth, and the like, and combinations thereof.

The term “dispersing agent” as used herein means a substance used to promote the disintegration and/or dissolution of tablet. Suitable dispersing agents include colloidal silicon dioxide, calcium silicate, magnesium trisilicate, silicon hydrogel and silica derivatives. A preferred dispersing agent as per the present invention is colloidal silicon dioxide.

The term “binders” as used herein means substances used to cause adhesion of powder particles in tablet granulations. Suitable binders include, by way of example and without limitation, lactose, starches such as corn starch, potato starch, modified starches, sugars, guar gum, pectin, wax binders, microcrystalline cellulose, methylcellulose, carboxymethylcellulose, hydroxypropylmethylcellulose, hydroxyethyl cellulose, hydroxypropyl cellulose, copolyvidone, so sodium alginate, acacia, alginic acid, tragacanth, carboxymethylcellulose sodium, ethyl cellulose, gelatin, liquid glucose, povidone, pregelatinized starch, and the like, and mixtures thereof.

The term “lubricant” as used herein means substances used to reduce friction during tablet compression. Suitable lubricants include, by way of example and without limitation, calcium stearate, magnesium stearate, zinc stearate, mineral oil, stearic acid, fumaric acid, palmitic acid, talc, carnauba wax, hydrogenated vegetable oils, mineral oil, polyethylene glycols, sodium stearyl fumarate, and the like, and combinations thereof.

The term “glidant” as used herein means agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, and the like, and combinations thereof.

The solid oral dosage forms, e.g., tablets, disclosed herein are optionally coated with an aqueous or non aqueous solution or dispersion of film forming agents. In one embodiment, the film coat is an aqueous moisture barrier. The coating solution comprises film forming polymers and one or more of plasticizers, opacifier, surfactant, anti-tacking agents, coloring agents and the like, and combinations thereof.

The coating is applied by solubilising or suspending the excipients in solvents such as isopropyl alcohol, water, acetone, ethanol, methylene chloride, and the like, and mixtures thereof.

In one embodiment, a composition comprises 22-27 wt % of maraviroc, 50-75 wt % of a diluent, 1-8 wt % of a disintegrant, optionally 0.5-2 wt % of a lubricant, optionally 0.1-4 wt % of a glidant and optionally 1-4 wt % of a coating material based on the total weight of the tablet, wherein the tablet is prepared by either direct compression or dry granulation by slugging.

In one embodiment, the ratio of disintegrant to dispersing agent is 1:0.25 to 1:1.

In one embodiment, the total weight of the tablet is over 1000 mg. In a specific embodiment, the tablet comprises 300 mg of maraviroc.

In one embodiment, the disintegration time of the oral dosage form is less than 2 minutes, specifically less than 1 minute when measured at 37° C.±2° C. by USP disintegration apparatus.

Solid oral compositions are prepared by direct compression or dry granulation.

A direct compression process for preparing maraviroc tablets comprises (i) dry mixing and blending maraviroc with a diluent, a disintegrant and colloidal silicon dioxide, (ii) lubricating the blend obtained in step (i) by adding a lubricant and finally (iii) compressing the lubricated blend of step (ii) into tablets or filling the lubricated blend into capsules.

In one embodiment, dry granulation comprises (i) sifting and blending maraviroc with a diluent, disintegrant and colloidal silicon dioxide; (ii) compressing the blended mixture of step (i) to form slugs; (iii) sizing the slugs to form granules; (iv) blending the granules with an additional excipient to form blended granules; and (v) compressing the blended granules of step (iv) in to tablets or filling the blended granules into capsules. In one embodiment, the additional excipient comprises a binder, a lubricant or a glidant.

Alternatively, dry granulation comprises compacting maraviroc, a diluent, a disintegrant and colloidal silicon dioxide in a roller compactor, and passing the compacts through a sieve such as an ASTM sieve #20 to obtain granules. The granules were lubricated and compressed into tablets on a rotary compression machine. The resulting tablets were optionally coated with Opadry.

In another embodiment, a tablet composition comprises, based on the total weight of the tablet, i) 20 wt % to 30 wt % of amorphous maraviroc having a particle size d₉₀ not more than 150 μm; ii) 0.5 wt % to 5 wt % of a colloidal silicon dioxide dispersing agent; iii) 40 wt % to 80 wt % of a diluent; and iv) 1 wt % to 8 wt % of a disintegrant; where in the tablet is prepared either by direct compression or dry granulation by slugging.

Maraviroc as used herein may take any of the forms selected from amorphous maraviroc, crystalline Form A, Form B, Form 1, Form 2, Form 3 or Form 4 of maraviroc, or combinations thereof. In one embodiment, the maraviroc is amorphous maraviroc.

In one embodiment, a solid oral composition comprises amorphous maraviroc having a particle size d₉₀ not more than 150 μm, specifically 5-100 μm, more specifically 10-80 μm.

If the particle size of amorphous maraviroc is less than 10 μm, process issues like sticking, poor flow, weight variation may occur. If the particle size of amorphous maraviroc is more than 150 μm, the dissolution rate may be hindered which ultimately affects in-vivo performance of the drug product.

Particle size can be reduced and/or controlled using techniques such as milling. A desired particle size of maraviroc is obtained by a suitable micronization technique known in the art such dry milling, wet milling, air jet milling, sieving, homogenizing using homogenizer, such as rotor-stator and/or high pressure homogenizer such as a MICROFLUIDIZER and the like. Micronized maraviroc provides good in vitro end release and in vivo bioavailability.

An unformulated active pharmaceutical ingredient as disclosed herein can have a particle size defined by the particle size distribution. The D₁₀, or 10^{th} volume percentile, is the size of particles below which 10% of the measured particle volumes, lie; the D₅₀, or 50^{th} volume percentile, is the size of particles below which 50% of the measured particle volumes, lie; and the D₉₀, or 90^{th} volume percentile, is the size of particles below which 90% of the measured particle volumes, lie. Particle size distributions can be determined using laser diffraction using a Malvern Mastersizer, for example.

Pharmaceutical composition comprising a therapeutically effective amount of maraviroc as disclosed herein is useful for treating HIV-1 infections.

Also included herein are methods of improving patient compliance by administering the dosage forms with favorable disintegration times as disclosed herein.

The invention is further exemplified with following examples which are not intended to limit the scope of the invention.

### EXAMPLES

### Examples 1-3

### Maraviroc Tablets Compositions Prepared by Direct Composition

| | | Qty per unit | | |
|---|---|---|---|---|
| | | | | Comparative |
| S. No | Ingredients | Example-1 | Example-2 | Example-3 |

Pre-mix

| | | | | |
|---|---|---|---|---|
| 1 | Maraviroc amorphous | 300.000 | 300.000 | 300.000 |
| 2 | Microcrystalline cellulose | 709.125 | 640.625 | 663.625 |
| 3 | Lactose monohydrate | 57.500 | 126.000 | 126.000 |
| 4 | Colloidal silicon dioxide | 23.000 | 23.000 | — |
| 5 | Sodium starch glycolate | 46.000 | 46.000 | 46.000 |

Final Mix/Lubrication

| | | | | |
|---|---|---|---|---|
| 6 | Magnesium stearate | 14.375 | 14.375 | 14.375 |

| | | | |
|---|---|---|---|
| Core Tablet Weight | 1150.000 | 1150.000 | 1150.000 |

Coating (15% w/w Suspension)

| | | | | |
|---|---|---|---|---|
| 7 | Opadry II Blue 85G20583, | 20.125 | 20.125 | 20.125 |
| 8 | Purified water | 114.040 | 114.040 | 114.040 |

| | | | |
|---|---|---|---|
| Total Tablet Weight | 1170.125 | 1170.125 | 1170.125 |
| Disintegration time | Less than 1 min | Less than 1 min | 3-4 min |

The disintegration time was measured at 37° C.±2° C. by USP disintegration apparatus.

### Manufacturing Process:

i) Maraviroc, microcrystalline cellulose, lactose, colloidal silicon dioxide and sodium starch glycolate were sifted through mesh #40.
ii) the materials of step no. (i) were loaded into a blender and mixed for 15 minutes.
iii) magnesium stearate was sifted through mesh #60 and added to the materials of step no. (ii) and blended for 5 minutes.
iv) the blend of step no. (iii) was compressed into tablets or filled into capsules.
v) the tablets of step no. (iv) were film coated using Opadry II Blue 85G20583.

### Dissolution Data for Examples 1-3:

Dissolution in 900 ml of 0.01N HCl, USP-I, 100 rpm

| | |
|---|---|
| Time | Cumulative % Drug release |

| in | Selzentry ® 300 mg | | | |
|---|---|---|---|---|
| min | (Innovator) | EX 1 | EX 2 | EX 3 |
| 5 | 62 | 56 | 66 | 38 |
| 10 | 80 | 77 | 85 | 62 |
| 15 | 88 | 89 | 94 | 76 |
| 20 | 92 | 95 | 96 | 87 |
| 30 | 95 | 98 | 96 | 93 |
| 45 | 97 | 98 | 97 | 96 |

Based on the dissolution data of Examples 1-3, it has been observed that, the maraviroc tablets of example 1 and 2 containing colloidal silicon dioxide exhibited an improved dissolution profile as compared to the pharmaceutical composition of comparative example 3.

### Example 4

### Solid Oral Compositions of Maraviroc Prepared by Dry Granulation by Slugging

| | | |
|---|---|---|
| S. No | Ingredients | Mg/tablet |

Intragranular

| | | |
|---|---|---|
| 1 | Maraviroc | 300.00 |
| 2 | Microcrystalline | 500.00 |
| | cellulose | |
| 3 | Corn starch | 126.00 |
| 4 | Croscarmellose sodium | 10.00 |
| 5 | Magnesium stearate | 6.00 |

Extragranular

| | | |
|---|---|---|
| 6 | Microcrystalline | 190.00 |
| | cellulose | |
| 7 | Colloidal silicon dioxide | 12.50 |
| 8 | Magnesium stearate | 5.50 |

| | |
|---|---|
| Total tablet weight | 1150.00 |

### Manufacturing Process:

i) Maraviroc, microcrystalline cellulose, corn starch, and croscarmellose sodium were sifted together through #40 mesh.
ii) magnesium stearate sifted separately through #60 mesh.
iii) material of step no .(i) & (ii) were mixed together for 10 minutes.
iv) the above material was slugged and the resulted slugs were milled using multimill or cone mill with 2 mm screen.
v) milled granules of step (iv) were sifted through #30 mesh completely.
vi) microcrystalline cellulose, magnesium stearate and colloidal silicon dioxide of extra granular portion were sifted together through #40 mesh, and added to the sifted granules of step (v) and blended for 10 minutes.
vii) the blend of step no. (vi) was compressed into tablets or filled into capsules.
viii) compressed tablets were optionally coated with Opadry II.

### Example 5

A biostudy was conducted to compare the formulation of Example 2 with the commercially available Selzentry® 300 mg tablet. The studies were open-label, balanced, randomized, two-treatment, two-period, two-sequence cross-over, single-dose bioequivalence studies, in 50 healthy human subjects. The study was conducted under fed and fasting conditions. Selzentry® is described as a film-coated 300 mg tablet containing dibasic calcium phosphate, magnesium stearate, microcrystalline cellulose, and sodium starch glycolate. The coating is Opadry II Blue.

In certain embodiments, the formulations described herein are bioequivalent to the reference listed dosage form (RLD).

The study duration was 11 days with a washout period of 11 days within both treatment periods. The study parameters determined were Cₘₐₓ, Tₘₐₓ, AUC₀₋ₜ, AUC_{0-inf}, t_{1/2}, and Kₑₗ.

The pharmacokinetic parameters for the fed study are given below:

| | Maraviroc-test according | Maraviroc-commercial |
|---|---|---|
| PK parameters | to present disclosure | dosage form |
| Tₘₐₓ (hr) | 3.68 ± 1.4441 | 3.675 ± 1.3087 |
| Cₘₐₓ (ng/mL) | 568.858 ± 350.6957 | 547.161 ± 384.8896 |
| AUC₀₋ₜ (ng h/mL) | 1934 ± 1194.3276 | 1708.992 ± 754.1851 |
| AUC_{0-inf} (ng h/ML) | 2255.734 ± 1257.2403 | 1954.676 ± 771.7304 |
| T_{1/2} (hr) | 5.653 ± 4.3898 | 4.249 ± 2.0422 |
| Kₑₗ (1/hr) | 0.198 ± 0.1254 | 0.201 ± .0936 |

As can be seen from the table, the test maraviroc formulation according to the present disclosure is bioequivalent to the commercially available product under fed conditions.

The pharmacokinetic parameters for the fasting study are given below:

| | Maraviroc-test according | Maraviroc-commercial |
|---|---|---|
| PK parameters | to present disclosure | dosage form |
| Tₘₐₓ (hr) | 2.981 ± 1.1880 | 2.965 ± 1.3131 |
| Cₘₐₓ (ng/mL) | 1035.898 ± 381.7004 | 1121.380 ± 402.6862 |
| AUC₀₋ₜ (ng h/mL) | 3573.856 ± 1195.4966 | 3498.488 ± 1115.9285 |
| AUC_{0-inf} (ng h/ML) | 3752.359 ± 1215.8244 | 3679.678 ± 1182.3191 |
| T_{1/2 (hr)} | 2.850 ± 0.0992 | 0.293 ± 0.1019 |
| Kₑₗ (1/hr) | 4.993 ± 3.0593 | 4.866 ± 2.5526 |

As can be seen from the table, the test maraviroc formulation according to the present disclosure is bioequivalent to the commercially available product under fasting conditions.

As used herein, a polymorph is a crystalline form of an active pharmaceutical ingredient, and includes crystalline polymorphs, amorphous forms, as well as solvate and hydrate forms, which are often referred to as pseudopolymorphs. Solvates are crystalline forms containing a stoichiometric or nonstoichiometric amount of solvent. A hydrate is a solvate wherein the solvent is water.

Amorphous forms are disordered forms that do not have a distinguishable crystalline lattice. Amorphous materials typically do not have sharp, well-defined reflections in their x-ray diffraction patterns, but rather a broad peak spanning a range of two-theta angles.

An active pharmaceutical ingredient can be used as a pharmaceutically acceptable salt. As used herein a “pharmaceutically acceptable salt” is a salt of an acidic or basic group that is non-toxic. Exemplary acids used to form pharmaceutically acceptable salts include containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, mesylate, saccharate, formate, benzoate, glutamate, methanesulfonate, ethanesulfonate, benzenesulfonate, p-toluenesulfonate and pamoate salts. Exemplary bases include sodium hydroxide, sodium carbonate, potassium hydroxide, aluminum hydroxide, calcium hydroxide, ethanolamine, diethanolamine, triethanolamine, tromethamine, N-methylglucamine and the like.

The dissolution properties of a dosage form can be tested by methods known in the art. An exemplary condition is a USP type 2 (paddle) apparatus at 50 rpm in 900 ml of phosphate buffer with pH 7.5 and at 37° C. Alternatively, a basket method may be employed. An alternative set of conditions is 37° C. for 2 hours in 0.1 M HCl, optionally followed by testing in a buffer pH 6.2.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A solid oral composition comprising maraviroc, a diluent, a disintegrant, and colloidal silicon dioxide as a dispersing agent, wherein the solid oral composition is prepared either by direct compression or dry granulation.

2. The solid oral composition according to claim 1, selected from a tablet, a capsule and a granule.

3. The solid oral composition according to claim 1, further comprising a binder, a lubricant, a glidant, or a combination thereof.

4. A solid oral composition comprising amorphous maraviroc having a particle size d₉₀ not more than 150 μm.

5. The solid oral composition according to claim 4, wherein the amorphous maraviroc preferably has a d₉₀ particle size of 5-100 μm.

6. The solid oral composition according to claim 1, wherein the diluent is selected from the group consisting of lactose, dibasic calcium phosphate, sucrose, glucose, mannitol, sorbitol, calcium carbonate, starch, microcrystalline cellulose, cellulose derivatives, Prosolv, and combinations thereof.

7. The solid oral composition according to claim 1, wherein the disintegrant is selected from croscarmellose sodium, crospovidone, polacrillin potassium, calcium silicate, carboxymethylcellulose sodium, sodium starch glycolate, starch, pregelatinised starch and combinations thereof.

8. The solid oral composition of claim 1, wherein the solid oral dosage form comprises 22-27 wt % of maraviroc, 50-75 wt % of the diluent, 1-8 wt % of the disintegrant, 0.5 wt % to 5 wt % of the colloidal silicon dioxide as a dispersing agent, optionally 0.5-2 wt % of a lubricant, and optionally 1-4 wt % of a coating material, based on the total weight of the tablet.

9. The solid oral composition of claim 1, in the form of a tablet comprising microcrystalline cellulose and lactose as the diluents, magnesium stearate as a lubricant and disintegrant selected from the group consisting of croscarmellose sodium, sodium starch glycolate, crospovidone, and polacrilin potassium.

10. The solid oral composition of claim 4 in the form of a tablet comprising, based on the total weight of the composition, i) 20 wt % to 30 wt % of the amorphous maraviroc having a particle size d₉₀ 5-100 μm; ii) 0.5 wt % to 5 wt % of colloidal silicon dioxide as a dispersing agent; iii) 40 wt % to 80 wt % of a diluent; and iv) 1 wt % to 8 wt % of a disintegrant.

11. The solid oral composition of claim 1, further comprising a film coating.

12. The solid oral composition of claim 1, in the form of a tablet composition comprising maraviroc, microcrystalline cellulose, lactose, colloidal silicon dioxide, magnesium stearate and one more disintegrants selected from croscarmellose sodium, sodium starch glycolate, crospovidone and polacrilin potassium.

13. A process for the preparation of tablet composition comprising maraviroc, comprising: (ia) dry mixing the maraviroc with a diluent, a disintegrant and colloidal silicon dioxide to form a dry mix, (iia) lubricating the blend with a lubricant, and (iiia) compressing the lubricated blend of step (iia) into tablets, or
(ib) sifting and blending the maraviroc with a diluent, a disintegrant and colloidal silicon dioxide, (iib) compressing the blended mixture of step (ib) to form slugs, (iiib) sizing the slugs to form granules; (ivb) blending the granules with at least one excipient selected from a binder, a lubricant, a dispersing agent, a disintegrant and a glidant; and finally (vb) compressing the granules of step (ivb) into tablets.

14. (canceled)

15. The composition according to claims 1, wherein the maraviroc is in the form of amorphous maraviroc, crystalline Form 1 of maraviroc phosphate, crystalline Form 2 of maraviroc phosphate, crystalline Form 3 of maraviroc phosphate, crystalline Form 4 of maraviroc phosphate, or a combinations thereof.

16. A method of improving patient compliance in a patient in need of treatment of HIV-1 comprising administering the oral dosage form of claim 1.
